(19) United States
(12) Patent Application Publication
(10) Pub. No.: US 2023/0338398 A1
(43) Pub. Date: Oct. 26, 2023

(54) USE OF DELTA-8-THC TO TREAT INFLAMMATORY AND AUTOIMMUNE DISEASES

(71) Applicant: UNIVERSITY OF SOUTH CAROLINA, Columbia, SC (US)

(72) Inventors: PRAKASH NAGARKATTI, COLUMBIA, SC (US); MITZI NAGARKATTI, COLUMBIA, SC (US); KHADIJA KAKAR, COLUMBIA, SC (US)

(21) Appl. No.: 18/105,511

(22) Filed: Feb. 3, 2023

Related U.S. Application Data
(60) Provisional application No. 63/306,151, filed on Feb. 3, 2022.
(60) Provisional application No. 63/323,581, filed on Mar. 25, 2022.

(51) Int. Cl. A61K 31/00; A61P 25/28; A61P 37/06; C12Q 1/6851

(52) U.S. Cl. CPC A61K 31/658; A61P 25/28; A61P 37/06; C12Q 1/6851; C12Q 2600/178; C12Q 2600/158

(57) ABSTRACT

The present disclosure is directed to a method for treating an autoimmune disease, the method comprising administering to a subject in need thereof a cannabinoid compound comprising delta-8-tetrahydrocannabinol.

### CROSS REFERENCE TO RELATED APPLICATIONS

This application claims filing benefit of U.S. Provisional Application Ser. No. 63/306,151, having a filing date of Feb. 3, 2022, and U.S. Provisional Application Ser. No. 63/323,581, having a filing date of Mar. 25, 2022, the entire contents of which are incorporated herein by reference.

### GOVERNMENT SUPPORT CLAUSE

This invention was made with government support under P01 AT003961, awarded by National Institutes of Health (NIH). The government has certain rights in the invention.

### SEQUENCE LISTING

The instant application contains a Sequence Listing which has been submitted electronically in XML file format and is hereby incorporated by reference in its entirety. Said XML copy, created on Jun. 8, 2023, is named USC-732_1583_SL.xml and is 10,633 bytes in size.

### BACKGROUND

Autoimmune diseases result in the body's immune responses attacking its own tissues causing prolonged inflammation and subsequent tissue damage. A broad spectrum of autoimmune diseases affects more than 25 million people. These diseases are chronic and can be life-threatening. It is estimated that autoimmune diseases are one of the top 10 leading causes of death in female children and women.

Multiple Sclerosis (MS) is a chronic debilitating autoimmune disease characterized by neuroinflammation leading to demyelination of neurons in the CNS and spinal cord. The incidence of MS is higher in women. Although the precise etiology of MS remains unclear, it is believed that genetic and environmental factors may promote MS. MS is caused by autoreactive T cells that belong to the Th1 and Th17 type. Such T cells, when activated by the antigens found in the myelin, cross the blood brain barrier (BBB) and enter the central nervous system CNS), and cause inflammation. Such neuroinflammation attracts other inflammatory cells, including macrophages, which cause demyelination, axonal damage, and paralysis. The primary treatment protocol for MS focuses solely on treating symptoms caused by MS.

What is needed in the art are clinically effective compositions and methods for treating autoimmune diseases.

### SUMMARY

In general, the present disclosure is directed to a method for treating an autoimmune disease, the method comprising administering to a subject in need thereof a cannabinoid compound comprising delta-8-tetrahydrocannabinol.

Numerous embodiments are further provided that can be applied to any aspect of the present disclosure and/or combined with any other embodiment described herein. For instance, in one embodiment, the autoimmune disease is multiple sclerosis. In another embodiment, the delta-8-tetrahydrocannabinol is administered to the subject at a dose of from about 0.01 mg/kg to about 10 mg/kg.

In another embodiment, the delta-8-tetrahydrocannabinol is administered to the subject daily for about 7 days to about 45 days.

In another embodiment, the subject is a human, a mouse, or a rat.

In another embodiment, the delta-8-tetrahydrocannabinol is administered intranasally, transdermally, or orally.

In another embodiment, the delta-8-tetrahydrocannabinol is substantially free of other psychotropic agent.

In another embodiment, the delta-8-tetrahydrocannabinol is substantially free of delta-9-tetrahydrocannabinol.

In another embodiment, methods disclosed herein further comprises administering to a subject in need thereof a pertussis toxin.

In another embodiment, the pertussis toxin is administered at a dose of from about 200 ng to about 400 ng.

In another embodiment, the pertussis toxin is administered from about 5 days to about 10 days before administration of the cannabidiol compound.

In another embodiment, the pertussis toxin is administered intraperitoneally.

In another embodiment, methods disclosed herein further comprises administering to a subject in need thereof an exogenous antigen.

In another embodiment, the exogenous antigen comprises Myelin oligodendrocyte glycoprotein (MOG35-55) peptide.

In another embodiment, the exogenous antigen comprises H-MEVGWYRSPFSRVVHLYRNGK-OH (SEQ ID NO: 1).

In another embodiment, the exogenous antigen is administered at a dose of from about 50 μg to about 200 μg.

In another embodiment, the exogenous antigen is administered intraperitoneally.

In another embodiment, the exogenous antigen is administered from about 5 days to about 10 days before administration of the cannabinoid compound.

In another embodiment, methods disclosed herein further comprises obtaining a biological sample from the subject; measuring expression level of at least one biomarker in a subject sample prior to and after administration of the cannabinoid compound; and comparing expression level of the biomarker.

In another embodiment, the cannabinoid compound increases the expression level of at least one biomarker.

In another embodiment, the cannabinoid compound decreases the expression level of at least one biomarker.

In another embodiment, the biomarker comprises a cytokine, a cell, a micro-RNA, or any combination thereof.

In another embodiment, the cytokine comprises IL-10, TGF-0, IL-17+, Foxp3, or any combination thereof.

In another embodiment, the cell comprises a cytotoxic T cell.

In another embodiment, the cytotoxic T cell is CD8+ T cell.

In another embodiment, the micro-RNA comprises miR-21, miR-27a, miR29a, miR-30a, miR-31, miR-146a, miR-155, miR-326, miR-let7, miR-130a, miR-181a, miR-328a, miR-448, or any combination thereof.

Each of the example aspects recited above may be combined with one or more of the other example aspects recited above in certain embodiments. For instance, all of the example aspects recited above may be combined with one another in some embodiments. As another example, any combination of two, three, four, five, or more of the twenty example aspects recited above may be combined in other embodiments. Thus, the example aspects recited above may be utilized in combination with one another in some example embodiments. Alternatively, the example aspects recited above may be individually implemented in other example embodiments. Accordingly, it will be understood that various example embodiments may be realized utilizing the example aspects recited above.

These and other features and aspects, embodiments and advantages of the present invention will become better understood with reference to the following description and appended claims.

### BRIEF DESCRIPTION OF THE DRAWINGS

A full and enabling disclosure of the present disclosure is set forth more particularly in the remainder of the specification, including reference to the accompanying figures, in which:

FIG. 1 depicts the disease model of Experimental Autoimmune Encephalomyelitis (EAE).

FIG. 2A depicts treatment of mice with EAE leads to attenuation of clinical score and decreases paralysis.

FIG. 2B depicts treatment of mice with EAE at day 22.

FIG. 3A depicts body weight loss of mice treated with EAE using Δ-8-THC.

FIG. 3B depicts body weight loss at day 22 of mice treated with EAE using Δ-8-THC.

FIG. 4A depicts Δ-8-THC treatment of EAE mice.

FIG. 4B depicts Δ-8-THC treatment of EAE mice leads to a decrease in brain-infiltrating CD4+ T cells.

FIG. 4C depicts Δ-8-THC treatment of EAE mice leads to a decrease in spleen-infiltrating CD4+ T cells.

FIG. 5A depicts brain-infiltrating pro-inflammatory macrophages in EAE mice following Δ-8-THC treatment.

FIG. 5B depicts brain-infiltrating pro-inflammatory macrophages decreased in EAE mice following Δ-8-THC treatment.

FIG. 5C depicts brain-infiltrating pro-inflammatory macrophages decreased in EAE mice following Δ-8-THC treatment.

FIG. 6A depicts Δ-8-THC treatment of EAE mice leads to a decrease in Th1 cells in the spleen and brain.

FIG. 6B depicts Δ-8-THC treatment of EAE mice leads to a decrease in Th1 cells in the spleen.

FIG. 6C depicts Δ-8-THC treatment of EAE mice leads to a decrease in Th1 cells in the brain.

FIG. 7A depicts Δ-8-THC treatment of EAE mice leads to a decrease Th1 cells expressing IFN-gamma in the spleens.

FIG. 7B depicts the percentage of Th1 cells expressing IFN-gamma in the spleens following Δ-8-THC treatment.

FIG. 8A depicts RORγt+ (Th17) cells and IL-17+ CD4+ T cells in the brains and spleens of EAE mice following treatment with Δ-8-THC.

FIG. 8B depicts RORγt+ (Th17) cells decrease in the brains of EAE mice following treatment with Δ-8-THC.

FIG. 8C depicts RORγt+ (Th17) cells decrease in the spleens of EAE mice following treatment with Δ-8-THC.

FIG. 8D depicts IL-17+ CD4+ T cells decrease in the brains of EAE mice following treatment with Δ-8-THC.

FIG. 9A depicts Δ-8-THC treatment leads to an increase in anti-inflammatory cytokine (IL-10) in the brain-infiltrating CD4+ T cells from EAE mice studied by qRT-PCR.

FIG. 9B depicts Δ-8-THC treatment leads to an increase in anti-inflammatory cytokine (TGF-β) in the brain-infiltrating CD4+ T cells from EAE mice studied by qRT-PCR.

FIG. 9C depicts Δ-8-THC treatment leads to an increase in Foxp3 in the brain-infiltrating CD4+ T cells from EAE mice studied by qRT-PCR.

FIG. 10 depicts miRNA expression profile of brain-infiltrating CD4+ T cells from EAE mice following treatment with Δ8-THC.

FIG. 11 depicts Δ-8-THC mediated regulation of miRNA signaling pathways in EAE mice.

FIG. 12 depicts Δ-8-THC mediated regulation of miRNA targets: pro- and anti-inflammatory response in EAE mice.

FIG. 13A depicts differential expression of miRNAs in brain infiltrating mononuclear cells following treatment of EAE mice with Δ-8-THC using miRNA sequencing.

FIG. 13B depicts miRNAs that are upregulated or downregulated in brain infiltrating mononuclear cells following treatment of EAE mice with Δ-8-THC.

FIG. 14A depicts genes targeted by miRNAs in brain infiltrating mononuclear cells of EAE mice following treatment with Δ-8-THC.

FIG. 14B depicts genes targeted by miR-193a-3p in brain infiltrating mononuclear cells of EAE mice following treatment with Δ-8-THC.

Repeat use of reference characters in the present specification and drawings is intended to represent the same or analogous features or elements of the present invention.

### DETAILED DESCRIPTION

Reference will now be made in detail to example embodiments of the disclosure. It is to be understood by one of ordinary skill in the art that the present disclosure is a description of exemplary embodiments only and is not intended as limiting the broader aspects of the present disclosure.

The present disclosure is generally directed to methods for treating an autoimmune disease or disorder by administering to the subject in need thereof at least one cannabinoid compound. The term “treating” as used herein refers to partially or completely alleviating, improving, relieving, inhibiting progression, and/or reducing incidence of one or more symptoms of an autoimmune disease or disorder.

In one embodiment, a subject in need thereof may be administered at least one cannabinoid compound. The term “cannabinoid compound” can refer to a 21-carbon-containing terpenophenolic compound produced by Cannabis species. For instance, the cannabinoid compound may be produced by Cannabis sativa. There are three main types of cannabinoids, such as herbal cannabinoids, synthetic cannabinoids, and endogenous cannabinoids. Cannabinoid compounds bind to evolutionarily conserved yet geographically and functionally distinct G protein-coupled receptors: cannabinoid receptor 1 (CB1) and cannabinoid receptor 2 (CB2). Cannabinoid receptor activation leads to a robust anti-inflammatory response, characterized by reduced antigen-presenting cell (APC) activation, a switch from a T helper 1 (Th1) phenotype to a T helper 2 (Th2) phenotype, direct induction of apoptosis in activated T cells, and induction of immunosuppressive cells such as Tregs and myeloid derived suppressor cells (MDSCs).

Cannabinoid compounds may be classified into subclasses, including Cannabidiol (CBD); Tetrahydrocannabinol (THC); Cannabigerols (CBG); Cannabichromenes (CBC); Cannabinol (CBN); Cannabicyclol (CBL); Cannabielsoin (CBE); and Cannabitriol (CBT).

In one embodiment, the cannabinoid compound may comprise CBD. CBD is a non-psychoactive cannabinoid that can trigger apoptosis in immune cells as well as act an anti-inflammatory agent. CBD has the following chemical formula: C₂₁H₃₀O₂.

In one embodiment, the cannabinoid compound may comprise THC. THC has the following chemical formula: C₂₁H₃₀O₂. It is well understood that THC is a key psychoactive cannabinoid present in cannabis. Depending on how it is derived, THC exists in various isomeric forms, including (+)trans-delta-8-THC (Δ-8-THC), (−)trans-delta-8-THC, (+)trans-delta-9-THC (Δ-9-THC), and (−)trans-delta-9-THC (Δ-9-THC).

Δ-8-THC is a structural isomer of Δ-9-THC with less psychoactive potency and better pharmacological effects. Δ8-THC is present in small quantities in cannabis. It exerts antispastic effects by binding to CB1 receptors located in the CNS as a partial agonist. Without wishing to be bound by theory, Δ-8-THC being less psychoactive compared to Δ-9-THC makes it a potential drug to treat an autoimmune disease clinically.

Δ-8-THC has the following chemical structure:

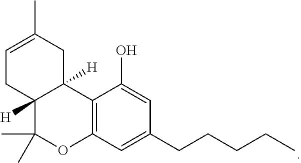 [SMILES: CCCCCc1cc(O)c2c(c1)OC(C)(C)[C@@H]1CC=C(C)C[C@@H]21]

In one embodiment, Δ-8-THC is substantially free of any psychotropic agent. In another embodiment, Δ-8-THC is substantially free of Δ⁹-THC.

The term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain example embodiments, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

In one embodiment, the cannabinoid compound may be administered at a dose of from about 0.01 mg/kg body weight to about 10 mg/kg body weight, such as from about 0.05 mg/kg body weight to about 5 mg/kg body weight, such as from about 0.1 mg/kg body weight to about 2.5 mg/kg body weight, such as from about 0.5 mg/kg to about 1 mg/kg, or any range therebetween. For instance, the cannabinoid compound (e.g., delta-8-THC) may be administered at a dose of at least about 0.8 mg/kg body weight. In another embodiment, the cannabinoid compound (e.g., delta-8-THC) may be administered at a dose of at least about 1 mg/kg body weight.

In one embodiment, a subject in need thereof may be administered at least one exogenous antigen. The exogenous antigen may be administered to induce immune tolerance in a subject suffering from or at risk of developing an autoimmune disease. The exogenous antigen may comprise a polypeptide, a carbohydrate, a nucleic acid, a lipid, a small molecule, or a combination thereof. In one embodiment, the exogenous antigen may comprise a polypeptide. The polypeptide may comprise from about 5 amino acids to about 100 amino acids, such as from about 10 amino acids to about 50 amino acids, such as from about 15 amino acids to about 45 amino acids, such as from about 20 amino acids to about 40 amino acids, such as from about 25 amino acids to about 35 amino acids, or any range therebetween. In one embodiment, the polypeptide comprises at least 20 amino acids.

Exogenous antigens described herein may be produced by various methods well known in the art. For instance, exogenous antigens may be obtained by extraction from isolated cells, by expression of a recombinant nucleic acid encoding the antigen, or by chemical synthesis. In one embodiment, recombinant technology may be utilized to produce the antigen polypeptide. In another embodiment, the antigen polypeptide may be produced by expression vectors encoding the polypeptide introduced into host cells (e.g., by transformation or transfection) for expression of the encoded antigen polypeptide.

The exogenous antigen may be selected from a group consisting of myelin basic protein, proteolipid protein, myelin oligodendrocyte glycoprotein (MOG), pancreatic beta cell antigen, and insulin. In one embodiment, the exogenous antigen may be a MOG polypeptide. For instance, the MOG polypeptide is an immunodominant 35-55 epitope of MOG (MOG35-55) peptide. Administration of MOG35-55 peptide in a subject produces anti-MOG antibodies that cause demyelination and a chronic Experimental Autoimmune Encephalomyelitis (EAE). Anti-MOG antibodies and the abnormal activation of encephalitogenic T cells upon MOG35-55 peptide binding destroying myelin sheath during Multiple Sclerosis.

In one embodiment, the exogenous comprises a peptide corresponding to the following sequence: H-MEVGWYRSPFSRVVHLYRNGK-OH (SEQ ID NO: 1).

The exogenous antigen disclosed herein may be administered at a dose of from about 25 μg to about 200 μg, such as from about 35 μg to about 175 μg, such as from about 40 μg to about 150 μg, such as from about 45 μg to about 150 μg, such as from about 50 μg to about 125 μg, or any range therebetween. For instance, the exogenous antigen (e.g., MOG35-55 peptide) may be administered at a dose of at least about 100 μg. In another embodiment, the exogenous antigen (e.g., MOG35-55 peptide) may be administered at a dose of at least about 150 μg.

In one embodiment, the exogenous antigen may interact with a cytotoxic agent. As used herein, “cytotoxic agent” refers to a compound or substance that inhibits or prevents a cellular function and/or causes cellular death. The cytotoxic agent may include, but is not limited to, a toxin, a radioactive isotope, a chemotherapeutic agent, a growth inhibitor agent, an enzyme, an antibiotic, or an anti-inflammatory agent. In one embodiment, the exogenous antigen may interact with a toxin. For instance, the toxin may be a protein toxin, a small molecule toxin, or an enzymatically active toxin of bacterial, fungal, plant, or animal origin. The protein toxin may include, but is not limited to, Pertussis toxin (PTX), CRM197, Diphtheria Toxin, Cholera holotoxin, Cholera Toxin B, Tetanus Toxin Fragment C, C. difficile Toxin B, P. aeruginosa Exotoxin A, diphtheria-A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-5), Momordica charantia inhibitor, curcin, crotin, Sapaonaria officinalis inhibitor, gelonin, mitogellin, restrictocin, phenomycin, enomycin, and tricothecenes.

In one embodiment, the protein toxin is Pertussis toxin. PTX is an exotoxin produced by Bordetella pertussis. According to methods disclosed herein, PTX may be utilized as a vaccine to protect against pertussis. PTX is useful as an adjuvant to enhance induction of autoimmune diseases (such as experimental autoimmune encephalomyelitis (EAE), experimental autoimmune orchitis, experimental autoimmune uveitis, etc.).

The cytotoxic agent may be administered at a dose of from about 100 ng to about 500 ng, such as from about 150 ng to about 450 ng, such as from about 200 ng to about 400 ng, such as from about 250 ng to about 350 ng, or any range therebetween. For instance, the cytotoxic agent (e.g., PTX) may be administered at a dose of at least about 100 ng. In one embodiment, the cytotoxic agent (e.g., PTX) may be administered at a dose of at least about 200 ng. In yet another embodiment, the cytotoxic agent (e.g., PTX) may be administered at a dose of at least about 400 ng.

The exogenous antigen and the cytotoxic agent may be administered concurrently or sequentially. For instance, the cytotoxic agent may be administered prior to administration of the exogenous antigen. In one embodiment, the cytotoxic agent may be administered after administration of the exogenous antigen. In another embodiment, the cytotoxic agent may be administered concurrently with the exogenous antigen.

Nevertheless, the exogenous antigen and/or the cytotoxic agent are administered prior to the administration of the cannabinoid compound. For instance, the exogenous antigen and/or the cytotoxic agent are administered from about 5 days to about 14 days before administration of the cannabinoid compound, such as from about 7 days to about 10 day before administration of the cannabinoid compound, or any range therebetween. In one embodiment, the exogenous antigen, the cytotoxic agent, or a combination thereof, is administered at least about 5 days before, at least about 6 days before, at least about 7 days before, at least about 8 days before, at least about 9 days before, at least about 10 days before, at least about 11 days before, at least about 12 days before, at least about 13 days before, at least about 14 days before administration of the cannabinoid compound.

The duration of therapy will continue for as long as medically indicated or until a desired therapeutic effect (e.g., those described herein) is achieved. For instance, subject can be treated until complete response, such as long as disease progression is delayed or inhibited. In one embodiment, the cannabinoid compound is administered daily for a period of about 1 day, about 2 days, about 3 days, about 4 days, about 5 days, about 6 days, about 7 days, about 10 days, about 14 days, about 21 days, about 28 days, about 6 weeks, about 8 weeks, or longer than 8 weeks following first administration. However, the course of treatment for any individual subject can be modified in clinical practice.

Methods disclosed herein are generally directed towards treating an autoimmune disease or disorder by administering to the subject in need thereof at least one cannabinoid compound. The term “treating” may refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition, e.g., an autoimmune disorder.

Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

An “autoimmune disease” or “autoimmune disorder” herein refers to a condition in which a subject's immune system attacks the body's own cells, causing tissue destruction. A subject may be diagnosed with an autoimmune disease using blood tests, cerebrospinal fluid analysis, electromyogram, or magnetic resonance imaging (MRI).

The autoimmune disease can include, but is not limited to, multiple sclerosis, arthritis, psoriasis, lupus, celiac disease, diabetes, mellitus type 1, Grave's disease, and inflammatory bowel disease. In one embodiment, the autoimmune disease is multiple sclerosis.

The term “subject” refers to any organism to which aspects of the disclosure can be administered, e.g., for experimental, diagnostic, prophylactic, and/or therapeutic purposes. Subjects to which embodiments of the disclosure can be administered include mammals, such as primates, for example, humans. For veterinary applications, a wide variety of subjects are suitable, e.g., livestock such as cattle, sheep, goats, cows, swine, and the like; poultry such as chickens, ducks, geese, turkeys, and the like; and domesticated animals, such as pets such as dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

As used herein, the term “administration” refers to introducing a substance (e.g., a cannabinoid compound, an exogenous antigen, a cytotoxic agent, etc.) into a subject. The administration thereof can be carried out in any convenient manner, including by aerosol inhalation, injection, ingestion, transfusion, implantation, or transplantation. For instance, the cannabinoid compound may be administered orally, subcutaneously, intravenously, or intratumoral. In this regard, “oral” administration can refer to administration into a subject's mouth; “subcutaneous” administration can refer to administration just below the skin; “intravenous” administration can refer to administration into a vein of a subject; and “intratumoral” administration can refer to administration within a tumor.

Pharmaceutical compositions disclosed herein may be formulated to be compatible with its intended route of administration. As used herein, “routes of administration” may include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition can be sterile and should be fluid to the extent that easy syringability exists. It can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. Prolonged absorption of the injectable compositions may be brought about by including in the composition an agent which delays absorption, for example, aluminum monostearate and gelatin.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Compositions for parenteral delivery, e.g., via injection, can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., corn oil) and injectable organic esters such as ethyl oleate. In addition, the composition can contain minor amounts of auxiliary substances such as wetting or emulsifying agents, pH buffering agents and the like that can enhance the effectiveness of the phenolic compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents.

In one embodiment, a therapeutically effective amount of the cannabinoid compound may be administered to the subject. The term “therapeutically effective amount” refers to those amounts that, when administered to a subject in view of the nature and severity of that subject's disease or condition, will have a desired therapeutic effect, e.g., an amount which will cure, prevent, inhibit, or at least partially arrest or partially prevent a target disease or condition. A therapeutically effective dose further can refer to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

A therapeutically effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

Methods disclosed herein further comprise obtaining a biological sample from a subject and measuring the presence or level of one or more biomarkers. As used herein, “obtaining a biological sample” refers to a process for directly or indirectly acquiring a biological sample from a subject. For instance, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, a “biopsy tissue” or a “tumor sample,” which refer to a sample of cells, tissues or fluids which is extracted from a subject, for example, in order to determine if the sample contains inflammation or to determine the gene expression profile or molecular profile of that tissue. The tissue or fluid may be examined to detect the presence or absence of one or more biomarkers of an autoimmune disease, including the presence of DNA and/or amino acid sequence mutations, molecular profile of the cells, and/or expression of the gene signature of cells.

The term “biomarker” may refer to mutations and/or molecules that can be evaluated in a biological sample (e.g., a biopsy tissue or a tumor sample) and are associated with a physical condition. For instance, biomarkers include expressed genes or their products (e.g., proteins) or antibodies to those proteins that can be detected from human samples, such as blood, serum, solid tissue, and the like, that is associated with a physical or disease condition. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins), and any complexes involving any such biomolecules, such as, but not limited to, a complex formed between an antigen and an autoantibody that binds to an available epitope on said antigen.

In one embodiment, the biomarker may comprise a cytokine, a cell, a micro-RNA, or any combination thereof. As used herein, “cytokine” refers to small, secreted proteins that regulate the intensity and duration of the immune response by affecting the immune cells differentiation process involving changes in gene expression by which a precursor cell becomes a distinct specialized cell type. For instance, the cytokine may comprise interleukin 10 (IL-100, transforming growth factor-beta (TGF-β), interleukin 17+(IL-17+), Foxp3, or any combination thereof.

In one embodiment, the biomarker may comprise a cell. For instance, the cell may be a cytotoxic T cell (e.g., CD8+ T cells) or T helper cell (e.g., CD4+ T cells). In one embodiment, the cell may comprise brain-infiltrating T cells, including CD4+, CD3+, CD45+, or CD8+.

In one embodiment, the biomarker may comprise a micro-RNA. miRNAs are understood to play a crucial role in autoimmune diseases. Previously, Δ-9-THC in combination with CBD has been found to downregulate miR-21a-5p, miR-31-5p, miR-122-5p, miR-146a-5p, miR-150-5p, miR-155-5p, and miR-27b-5p while upregulating miR-706-5p and miR-7116.

Interestingly, the present disclosure has found Δ-8-THC regulates a distinct set of miRNAs compared to Δ-9-THC. For instance, the microRNA (miRNA) may include, but is not limited to, miR-6538, miR-6845-3p, miR6946-3p, miR-193a-3p, miR-3547-3p, miR-701-3p, miR-7043-5p, miR-3079-5p, mi-7091-3p, miR-7042-5p, or any combination thereof (Table 1).

TABLE 1
miRNA Sequences

| Name | Sequence | SEQ ID NO |
|---|---|---|
| miR-6538 | CGCGGGCUCCGGGGCGGCG | 2 |
| miR-6845-3p | CCUCUCCUCCCUGUGCCCCAG | 3 |
| miR-6946-3p | UUUCUUCUUAGACAUGGCAACG | 4 |
| miR-193a-3p | AACUGGCCUACAAAGUCCCAGU | 5 |
| miR-3547-3p | UGAGCACCACCCCUCUCUCAGAU | 6 |
| miR-701-3p | UAUCUAUUAAAGAGGCUAGC | 7 |
| miR-7043-5p | UGUGAAAGCAGAGAGGCAUUUUU | 8 |
| miR-3079-5p | UUUGAUCUGAUGAGCUAAGCUGG | 9 |
| miR-7091-3p | AGUGGCUUCUGUCGUCUCUAG | 10 |
| miR-7042-5p | UAGAGACAGCAGAAGGGCCAC | 11 |

According to the present disclosure, delta-8-THC may advantageously mediate regulation of microRNA target genes, including pro- and anti-inflammatory response in EAE. In one embodiment, microRNA may regulate target genes including, but not limited to, Sox6, Cdkn1a, Psma8, Msantd3, Crispld2, Muc20, AANAT, AARD, ABCC1, ABCC12, ABCD4, ABCA10, ABCD2, ABHD17A, AC0648741, AADACL3, AAK1, ABCB10, ABCB8, ABI2, ABHD14B, ACOT11, ACSBG1, ACSBG2, ACSL1, AC006372.1, RP11-180C1.1, ZNF286A, HIST2H4B, TFRC, KIAA0087, RAB30, CDR2, TMSB15B, FTCDNL1, C1QL3, POLE3, CPN2, TRIML2, RP11-650K20.3, GRIN2B, ZNF776, C5orf55, ZNF544, ZNF345, AL590452.1, CTF1, KIAA1549L, or a combination thereof. Targeted gene regulation may be measured utilizing methods well known in the art. For instance, gene expression may be measured using miRNA sequencing (miRNAseq).

Methods disclosed herein may be beneficial for treating an autoimmune disorder. The expression level of a biomarker may be measured prior to and after administration of at least one compound disclosed herein. For instance, microRNA levels may be measured by any method well known in the art prior to and after administration of at least one compound disclosed herein.

In one embodiment, the expression level of a biomarker may be decreased by more than about 5%, 6%, 7%, 8%, 9%, 10%, 11%, 12%, 13%, 14%, 15%, 20%, 25%, 30%, 40%, 50%, 60%, 70%, 80%, 90%, 95%, or 100% compared to a controlled sample.

In one embodiment, the expression level of a biomarker may be increased by more than about 5%, 6%, 7%, 8%, 9%, 10%, 11%, 12%, 13%, 14%, 15%, 20%, 25%, 30%, 40%, 50%, 60%, 70%, 80%, 90%, 95%, or 100% compared to a controlled sample.

Following treatment based on methods disclosed herein, a subject may experience a reversal of body weight loss. For instance, the subject may gain or maintain body weight. In one embodiment, a subject may gain from about 14 g to about 50 g body weight, such as from about 15 g to about 25 g body weight, or any range therebetween.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Example 1

Example 1 discusses various methods and procedures and provides exemplary embodiments that may be understood in conjunction with the Drawings and Description provided herein.

### Methods

### Animal Models and Husbandry

6-8 weeks old Female C57BL/6 mice were purchased from Jackson Laboratory (Bar Harbor, ME). Animals colonies were maintained in accordance with Federal regulations and guidelines set by the animal facility of the University of South Carolina Institutional Animal Care and Use Committee (IACUC). Mice were housed in a single room under controlled temperature (22° C.), identical SPF, 50% relative humidity, and photoperiods (12:12 hours light/dark cycle). Animals were allowed unlimited access (ad libitum feeding) to autoclaved drinking water and a standard irradiated soy-free mouse chow. The health of the animals was monitored daily by the veterinarian in the animal facility.

### Reagents

The following reagents were used during the experiments and purchased as follows: Delta-8-THC was from Cayman Chemical (Ann Arbor, MI); Myelin oligodendrocyte glycoprotein (MOG35-55) peptide and H-MEVGWYRSPFSRVVHLYRNGK-OH (SEQ ID NO: 1) were from PolyPeptide Laboratories (San Diego, CA); pertussis toxin was purchased from List Biological Laboratories (Campbell, CA); Mycobacterium tuberculosis (strain H37Ra) and complete Freund's adjuvant were purchased from Difco (Detroit, MI); red blood cell (RBC) lysis buffer and β-mercaptoethanol were purchased from Sigma-Aldrich (St. Louis, MO); Neural Tissue Dissociation Kit (P) was purchased from Miltenyi Biotech Inc. (Auburn, CA); percoll was purchased from GE Healthcare Life Sciences (Pittsburgh, PA); RPMI 1640,1-glutamine, HEPES, phosphate-buffered saline, and fetal bovine serum (FBS) were from VWR (West Chester, PA); Annexin V/-PI apoptosis kit (Biolegend, San Diego, CA). EasySep PE selection kit (Stemcell Technologies, Cambridge, MA, USA). SsoAdvanced™ Universal SYBR® Green Supermix (Bio-Rad, Hercules, CA, USA), miRNeasy Mini Kit, miScript II RT Kit (Qiagen, Valencia, CA).

### Experimental Autoimmune Encephalomyelitis (EAE) Induction, Delta-8-THC Administration, and Clinical Assessment

EAE is a model for human Multiple Sclerosis. EAE was induced in 6-8 weeks old Female C57BL/6 mice through subcutaneous immunization with 100 μl of 150 μg MOG35-55 peptide (PolyPeptide Laboratories San Diego, CA, USA) along with heat killed Mycobacterium Tuberculosis 8 mg/ml (strain H37Ra) (BD, Franklin Lakes, NJ, USA), emulsified in PBS and complete Freund's adjuvant (CFA) (Fisher, Hampton, NH, USA). Mice also received two subsequent doses of 200 and 400 ng of pertussis toxin intraperitoneally on day 0 and 2, respectively (List Biological Laboratories, Campbell, CA, USA). Beginning on day 10, mice received a 100 μL intraperitoneal injection containing either a vehicle (sterile corn oil (CO) with 2% DMSO v/v) or a treatment suspension (10 mg/kg delta-8-THC in sterile CO with 2% DMSO v/v) every day till the end of the experiment. The control mice received the appropriate vehicle. On the appearance of the clinical symptoms, the animals were provided with food and water (Boost and Hydrogel) in the cage bedding to ensure their access to essential nutrients. During the experiment, animals were weighed for weight change and scored for disease progression on a daily basis. The mean body weight and clinical scores were calculated daily for each group. Clinical scores were recorded as follows: 0, healthy; 1, tail atony; 2, partial paralysis of hind limbs; 3, complete paralysis of hind limbs in combination with partial front limb paralysis; 4, tetraplegia; 5, moribund.

### Isolation of Immune Cells

On day 26, spleens were harvested from Vehicle and delta-8-THC-treated groups and were processed immediately to prepare single cell suspensions. Spleens were mechanically dissociated, and after RBC lysis the remaining cells were flittered through 70 μm filters. Animals were perfused with heparinized PBS to get rid of blood. Whole brain tissues were isolated and single cell suspensions were prepared using neural tissue dissociation kits (Miltenyi Biotech, Auburn, CA, USA) according to manufacturer instructions. The mononuclear cells were isolated by RBC lysis and using 33% Percoll gradient separation.

### Cell Culture

Immune cells from brain and splenocytes were cultured for 24 hours in complete RPMI containing 10% Heat inactivated Fetal Bovine Serum (FBS), 10 mM HEPES, 50 μM β-mercaptoethanol, 10 mM 1-glutamine, and 100 μg/ml penicillin/streptomycin at 37° C., 5% CO₂, 95% humidity. After 24 hours of culture, the supernatants were collected from cell culture for ELISA.

### CD4⁺ T Cell Selection

Mononuclear cells isolated from brain were labeled with Phycoerythrin-conjugated (PE-Conjugated) anti-CD4 antibody purchased from Biolegends. Then CD4+ cells were immunomagnetically selected with EasySep PE-positive selection kit according to the manufacturer's instructions (StemCell Technologies, Vancouver, BC). The purity of the cells was measured by flow cytometry to >90%.

### Flow Cytometry

BD FACSCelesta flow cytometer was used to quantify the phenotypes of immune cells from brain and spleens. Cells were stained with fluorescently labeled monoclonal antibodies purchased from Biolegend (San Diego, CA). The data obtained from flow cytometer was analyzed on FlowJo software.

RNA Isolation and cDNA Synthesis

To analyze gene expression, total RNA was purified from brain infiltrating CD4⁺ cells using miRNeasy micro kit according to the manufacturer's instructions. RNA purity and concentration were measured by using Nanodrop spectrophotometer from Thermoscientific. Next, the expression profiling of miRNAs using the Affymetrix GeneChip miRNA 4.0 array platform was performed. The qRT-PCR using SYBR Green Universal PCR Master Mix (Bio-Rad) in 96-well optical-reaction plates capped with optical adhesive covers (Applied Biosystems, Foster City, California, USA) was also performed, and reactions were run on a CFX96 Real-time PCR System (Bio-Rad). Housekeeping gene GAPDH was used in this study to normalize the expression of IL-10, Foxp3, and TGF-β. The results obtained from qRT-PCR were calculated using delta delta Ct (ΔΔCt) method.

### Statistical Analysis

The data were expressed as mean±SEM calculated using GraphPad Prism 9 (GraphPad Inc, La Jolla, CA) and the data sets for all experiments represent three to four experimental replicates per group. A Student's t-test for paired analyses or one- or two-way ANOVA for multiple group analyses were used to calculate the significance between the group. (*) p-value≤0.05 and (**)≤0.005 were considered statistically significant. Mann-Whitney U-test was performed to evaluate the clinical score of animals used in experiments.

### Example 2

Example 2 discusses various results provided in the drawings and described herein are meant to be exemplary and are not intended to limit the methods and compositions to modifications or alternatives as would be understood by a person of ordinary skill in the field of endeavor.

To test the anti-inflammatory properties of Δ-8-THC, a murine model of MS called Experimental Autoimmune Encephalomyelitis (EAE) was used. Treatment of C57BL/6 mice with Δ-8-THC (10 mg/kg b.w.) intraperitoneally caused a significant amelioration of EAE as indicated by a highly significant reduction in disease clinical scores and an increase in body weight. Δ-8-THC treatment also caused a decrease in infiltrating CD4+ T cells and an increase in anti-inflammatory molecules such as IL-10 and TGF-β. miRNA microarray analysis of CD4+ T cells isolated from the brain disclosed that the A8-THC treatment downregulated miR-21, miR-27a, miR-29a, miR-30a, miR-31, miR-146a, miR-155, and miR-326 while upregulating miR-let7, miR-130a, miR-181a, miR-328a and miR-448. Through pathway analysis, it was found that the majority of the downregulated miRNAs targeted molecules involved in apoptosis, migration, and cell cycle arrest, such as BCL7, MAP2K1/2, and CDKN2B, as well as promoted anti-inflammatory molecules, including DPEP2 and Smad1/2. Collectively, all of these findings demonstrated that Δ8-THC treatment can attenuate EAE potentially through modulation of the miRNA profile in the brain-infiltrating T cells, leading to decreased neuroinflammation. These studies suggest for the first time that Δ-8-THC can be used to treat a variety of inflammatory and autoimmune diseases, and because Δ-8-THC is less psychoactive than Δ-9-THC, it is more relevant for clinical use.

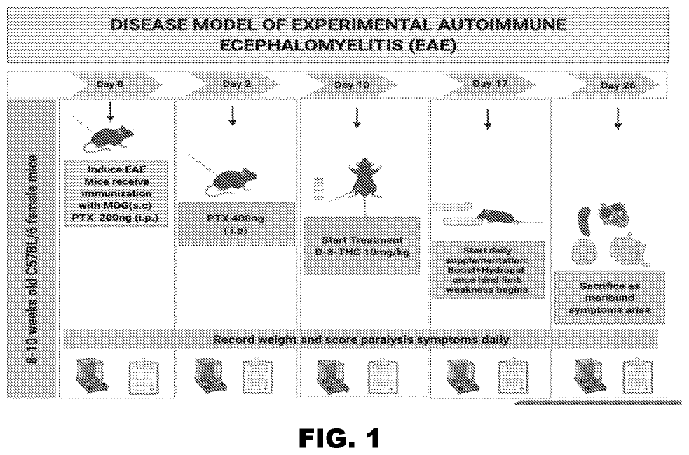

FIG. 1 shows treatment of mice with Experimental Autoimmune Encephalomyelitis (EAE), a model for human MS, leads to attenuation of clinical score and decreases paralysis. Control mice exhibiting EAE show paralysis and weight loss, whereas mice treated with Δ-8-THC attenuated paralysis and weight loss.

Scoring of paralysis symptoms key:
1=flat tail
2=weakness or partial paralysis of hind limb
3=complete paralysis of hind limbs or partial paralysis front limbs
4=Tetraparalysis
5=Moribund
6=Death

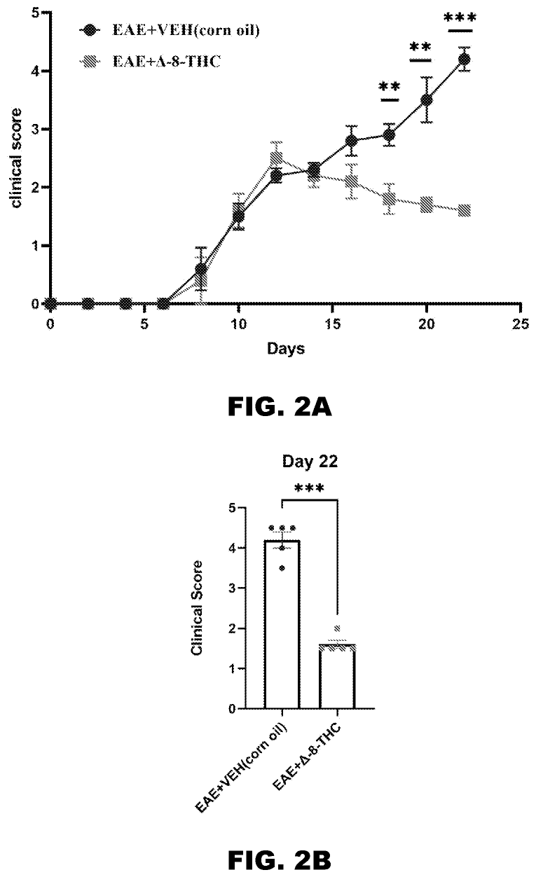

Mice were immunized with MOG35-55 to induce EAE and were treated with the vehicle or Δ8-THC. As depicted in FIG. 2A, mice treated with Δ-8-THC exhibited decreased levels of paralysis when compared to the control mice.

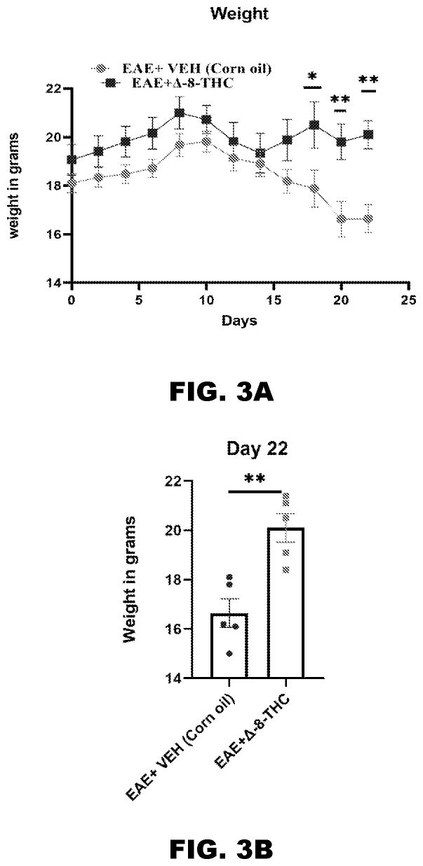

Mice were immunized with MOG to induce EAE and were treated with the vehicle or Δ8-THC. As depicted in FIG. 3A, control mice with EAE loss weight while mice treated with Δ8-THC gain weight. As such, these data demonstrate that treatment of mice with EAE using Δ8-THC leads to a reversal in body weight loss.

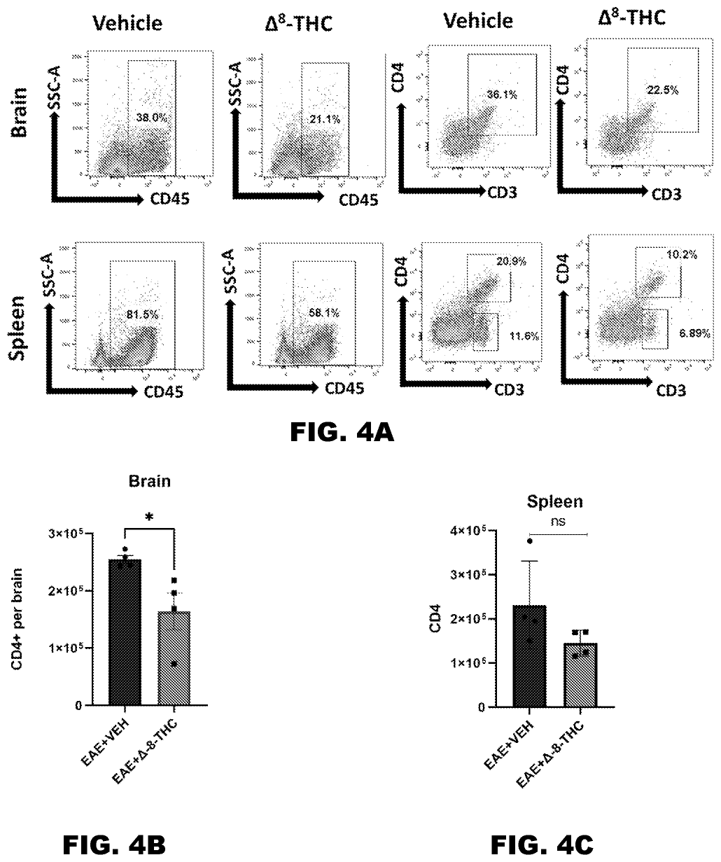

Further, mice treated with Δ-8-THC led to a decrease in the percentage of CD4+ T cells measured by flow cytometry in both the brain and spleens (FIG. 4A).

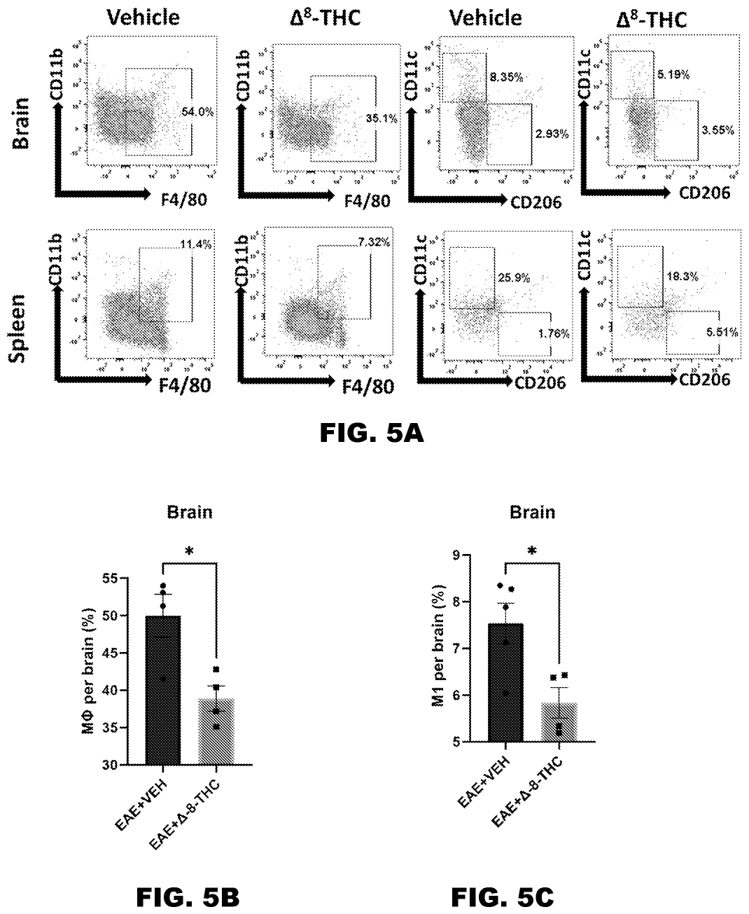

EAE mice treated with Δ-8-THC led to a decrease in the percentage of macrophages both in the brain and spleens (FIG. 5A). As such, brain-infiltrating pro-inflammatory macrophages are decreased in EAE mice following Δ8-THC treatment.

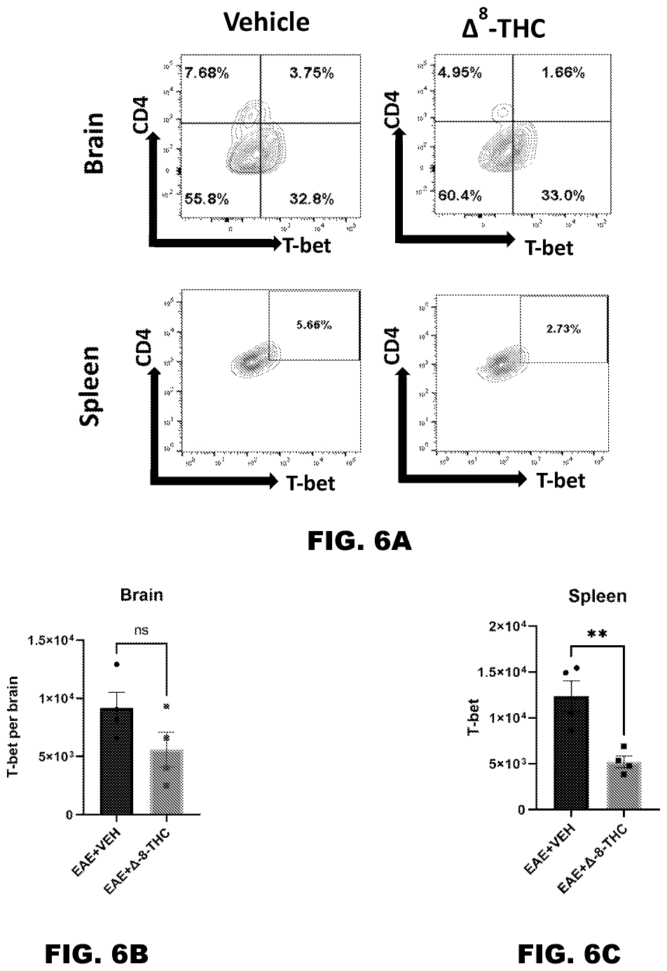

The treatment of EAE mice with Δ-8-THC led to a decrease in the percentage of these cells in the spleen but not in the brain (FIG. 6A). As depicted in FIG. 6B, Δ-8-THC treatment of EAE mice decreased Th1 cells in the spleens measured based on the expression of T-bet, which is a marker for inflammatory Th1 cells (FIG. 6B; FIG. 6C).

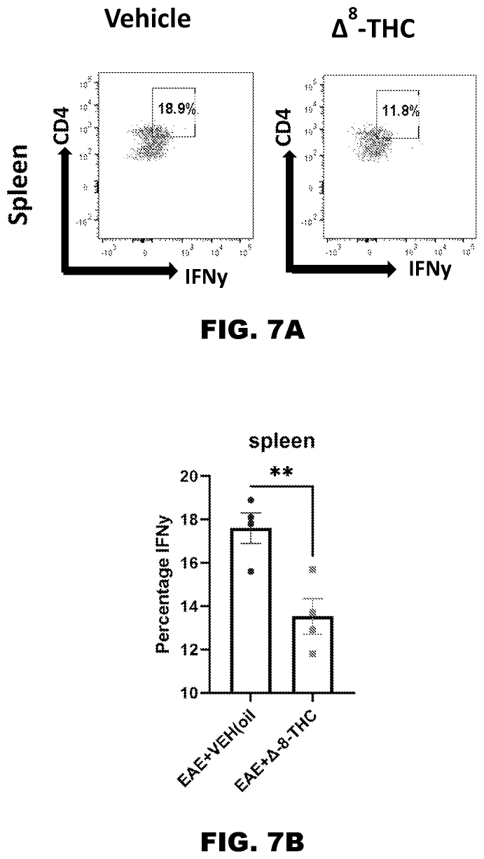

Following treating EAE mice with Δ-8-THC, Th1 cells expressing IFN-gamma in the spleens were decreased (FIG. 7A).

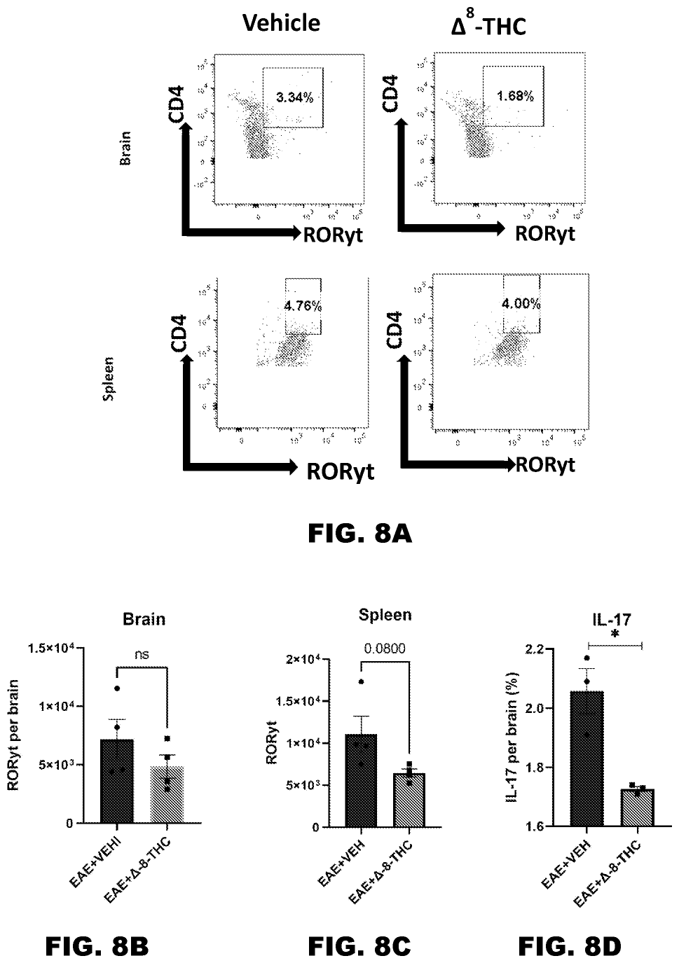

Expression of RORγt+ (Th17) cells and IL-17+ CD4+ T cells decreased in the brains and spleens of EAE mice following treatment with Δ8-THC (FIG. 8A). As such, the percentage of IL-17 producing inflammatory Th17 cells was decreased in EAE mice following Δ-8-THC treatment (FIG. 8D).

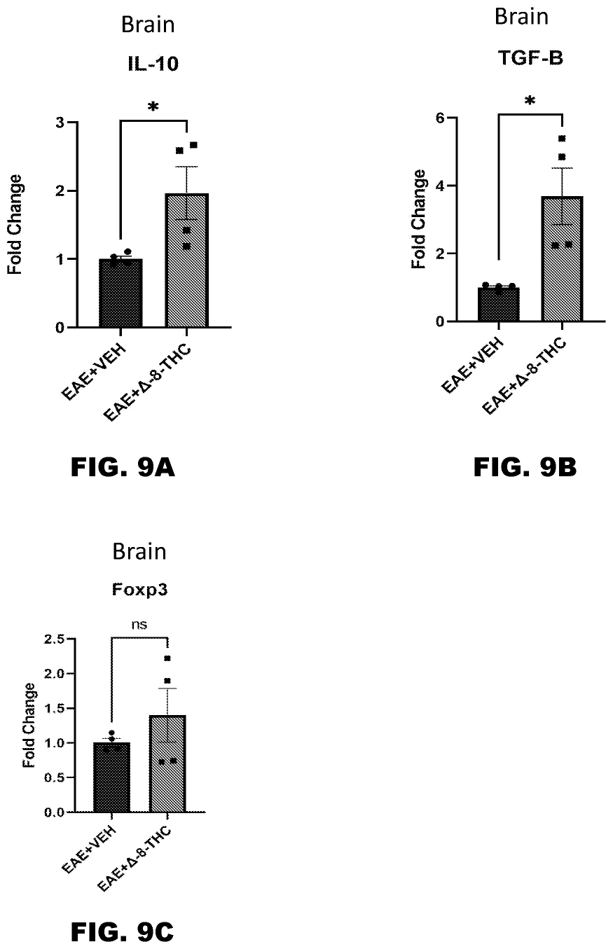

Δ-8-THC treatment increased anti-inflammatory cytokine expression (e.g., IL-10 and TGF-Beta) in the brain-infiltrating CD4+ T cells from EAE mice studied by qRT-PCR (FIG. 9A). EAE mice were treated with vehicle or Δ-8-THC. Δ-8-THC treatment led to an increase in anti-inflammatory cytokine (IL-10 and TGF-Beta) in the brain-infiltrating CD4+ T cells, suggesting that Δ-8-THC may suppress EAE through induction of IL-10 and TGF-beta.

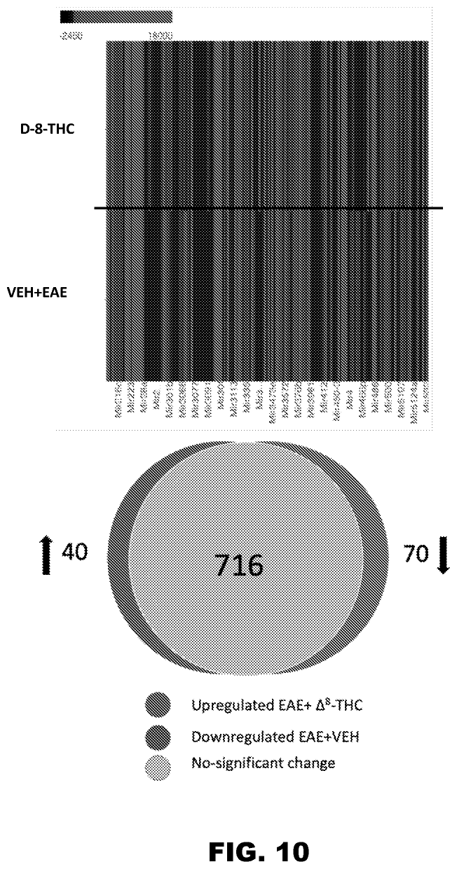

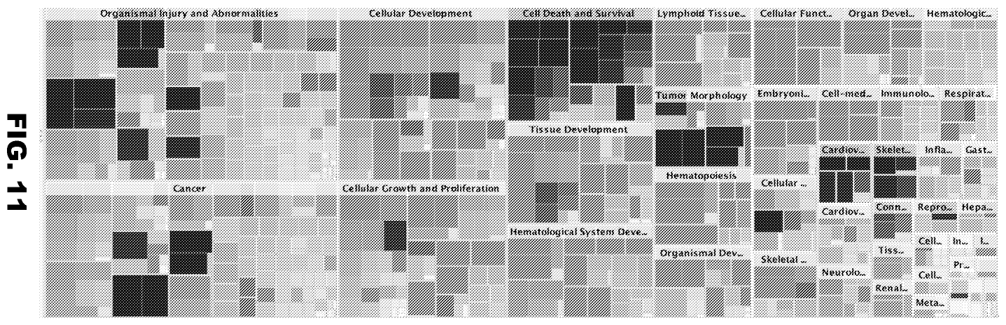

Interestingly, miR expression profile of brain-infiltrating CD4+ T cells from EAE mice following treatment with Δ-8-THC shows a distinct signature profile when compared to the controls (FIG. 10). Without wishing to be bound by theory, Δ-8-THC mediated regulation of miRNA signaling pathways in EAE mice suggests Δ-8-THC may act through a distinct pathway compared to Δ-9-THC (FIG. 11).

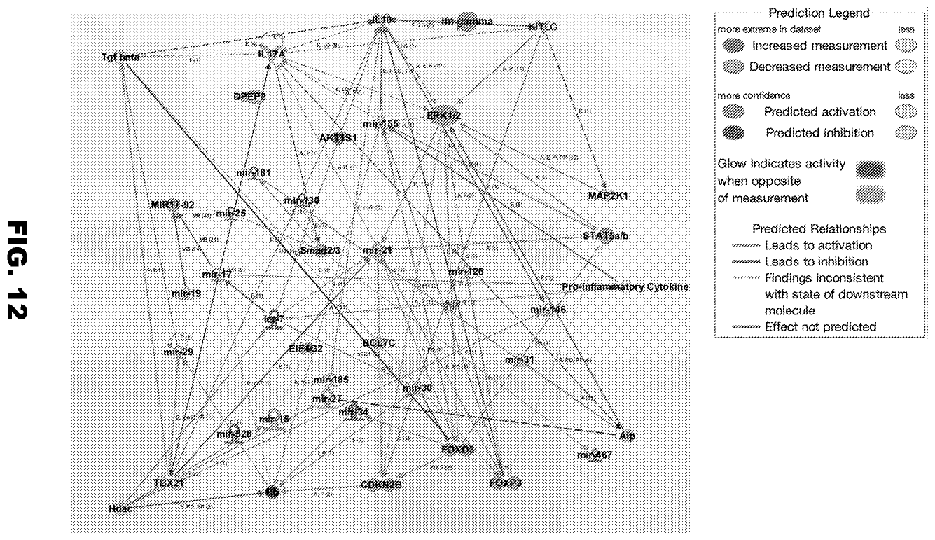

Δ-8-THC mediated regulation of miRNA targets: pro- and anti-inflammatory response in EAE mice (FIG. 12). Pathway analysis suggests Δ-8-THC may be utilized to regulate miRNAs that target various immunological pathways.

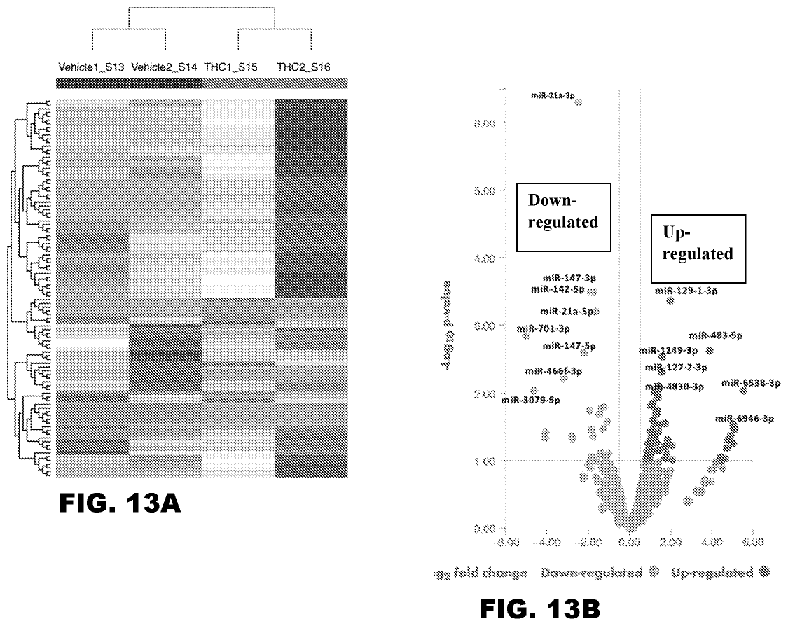

miRNA sequencing (miRNASeq) shows differential expression of miRNAs in brain infiltrating mononuclear cells following treatment of EAE mice with Δ-8-THC (FIG. 13A). FIG. 13B depicts that Δ-8-THC induces a unique miRNA signature profile distinct from Δ-9-THC.

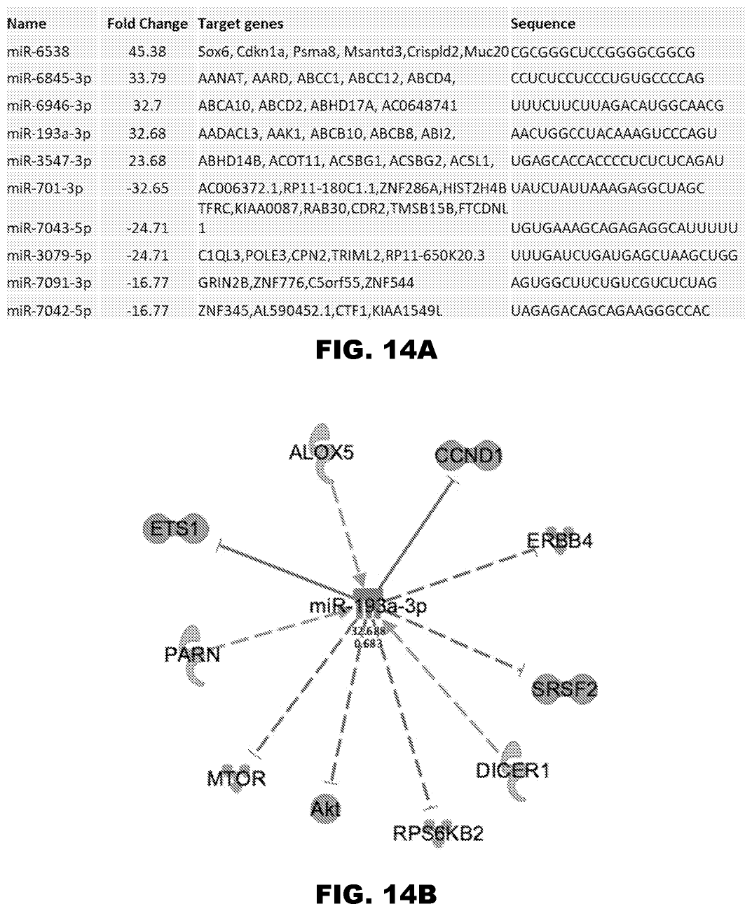

miRNASeq revealed differential expression of miRNAs in brain infiltrating mononuclear cells of EAE mice following treatment with Δ-8-THC (FIG. 14A).

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A method for treating an autoimmune disease, the method comprising administering to a subject in need thereof a cannabinoid compound comprising delta-8-tetrahydrocannabinol.

2. The method of claim 1, wherein the autoimmune disease is multiple sclerosis.

3. The method of claim 1, wherein the delta-8-tetrahydrocannabinol is administered to the subject at a dose of from about 0.01 mg/kg to about 10 mg/kg.

4. The method of claim 1, wherein the delta-8-tetrahydrocannabinol is administered to the subject daily for about 7 days to about 45 days.

5. The method of claim 1, wherein the subject is a human, a mouse or a rat.

6. The method of claim 1, wherein the delta-8-tetrahydrocannabinol is administered intranasally, transdermally, or orally.

7. The method of claim 1, wherein the delta-8-tetrahydrocannabinol is substantially free of additional psychotropic agent.

8. The method of claim 1, wherein the delta-8-tetrahydrocannabinol is substantially free of delta-9-tetrahydrocannabinol.

9. The method of claim 1, further comprises administering to a subject in need thereof a pertussis toxin.

10. The method of claim 9, wherein the pertussis toxin is administered at a dose of from about 200 ng to about 400 ng.

11. The method of claim 9, wherein the pertussis toxin is administered from about 5 days to about 10 days before administration of the cannabidiol compound.

12. The method of claim 9, wherein the pertussis toxin is administered intraperitoneally.

13. The method of claim 1, further comprises administering to a subject in need thereof an exogenous antigen.

14. The method of claim 13, wherein the exogenous antigen comprises Myelin oligodendrocyte glycoprotein (MOG35-55) peptide.

15. The method of claim 13, wherein the exogenous antigen comprises H-MEVGWYRSPFSRVVHLYRNGK-OH (SEQ ID NO: 1).

16. The method of claim 13, wherein the exogenous antigen is administered at a dose of from about 50 μg to about 200 μg.

17. The method of claim 13, wherein the exogenous antigen is administered intraperitoneally.

18. The method of claim 13, wherein the exogenous antigen is administered from about 5 days to about 10 days before administration of the cannabinoid compound.

19. The method of claim 1, further comprising:
obtaining a biological sample from the subject;
measuring expression level of at least one biomarker in a subject sample prior to and after administration of the cannabinoid compound; and
comparing expression level of the biomarker.

20. The method of claim 19, wherein the cannabinoid compound increases the expression level of at least one biomarker.

21. The method of claim 19, wherein the cannabinoid compound decreases the expression level of at least one biomarker.

22. The method of claim 19, wherein the biomarker comprises a cytokine, a cell, a micro-RNA, or any combination thereof.

23. The method of claim 22, wherein the cytokine comprises IL-10, TGF-β, IL-17+, Foxp3, or any combination thereof.

24. The method of claim 22, wherein the cell comprises a cytotoxic T cell.

25. The method of claim 22, wherein the cytotoxic T cell is CD8+ T cell.

26. The method of claim 22, wherein the micro-RNA comprises miR-21, miR-27a, miR29a, miR-30a, miR-31, miR-146a, miR-155, miR-326, miR-let7, miR-130a, miR-181a, miR-328a, miR-448, or any combination thereof.


SEQUENCE LISTING (11 sequences)

SEQ ID NO: 1 - protein, 21 aa, synthetic construct
MEVGWYRSPFSRVVHLYRNGK

SEQ ID NO: 2 - RNA, 19 nt, Mus sp.
  source 1..19: mol_type = genomic RNA
cgcgggctccggggcggcg

SEQ ID NO: 3 - RNA, 21 nt, Mus sp.
  source 1..21: mol_type = genomic RNA
cctctcctccctgtgccccag

SEQ ID NO: 4 - RNA, 22 nt, Mus sp.
  source 1..22: mol_type = genomic RNA
tttcttcttagacatggcaacg

SEQ ID NO: 5 - RNA, 22 nt, Mus sp.
  source 1..22: mol_type = genomic RNA
aactggcctacaaagtcccagt

SEQ ID NO: 6 - RNA, 23 nt, Mus sp.
  source 1..23: mol_type = genomic RNA
tgagcaccacccctctctcagat

SEQ ID NO: 7 - RNA, 20 nt, Mus sp.
  source 1..20: mol_type = genomic RNA
tatctattaaagaggctagc

SEQ ID NO: 8 - RNA, 23 nt, Mus sp.
  source 1..23: mol_type = genomic RNA
tgtgaaagcagagaggcattttt

SEQ ID NO: 9 - RNA, 23 nt, Mus sp.
  source 1..23: mol_type = genomic RNA
tttgatctgatgagctaagctgg

SEQ ID NO: 10 - RNA, 21 nt, Mus sp.
  source 1..21: mol_type = genomic RNA
agtggcttctgtcgtctctag

SEQ ID NO: 11 - RNA, 21 nt, Mus sp.
  source 1..21: mol_type = genomic RNA
tagagacagcagaagggccac